(19) United States
(12) Patent Application Publication
(10) Pub. No.: US 2007/0072840 A1
(43) Pub. Date: Mar. 29, 2007

(54) POLYMORPHIC FORMS OF QUETIAPINE

(76) Inventors: Bhargav PANDYA, Ahmedabad (IN); Ram Chander Aryan, New Delhi (IN)

Correspondence Address: RANBAXY INC., 600 COLLEGE ROAD EAST, SUITE 2100, PRINCETON, NJ, 08540, US

(21) Appl. No.: 11/421,018

(22) Filed: May 30, 2006

(30) Foreign Application Priority Data
May 30, 2005 (IN) 1389/DEL/2005

(51) Int. Cl. A61K 31/554; C07D 417/02

(52) U.S. Cl. 514/211.13; 540/551

(57) ABSTRACT

The invention relates to processes for the preparation of polymorphic forms of salts of quetiapine. More particularly, it relates to the preparation of polymorphic forms of quetiapine hydrochloride designated as Form A, B, and C. The invention also relates to pharmaceutical compositions that include the Form A, Form B, and Form C and use of the compositions for treating schizophrenia, acute manic episodes associated with bipolar I disorder. The invention also relates to the preparation of an oxalate salt of quetiapine and maleate salt of quetiapine.

### FIELD OF THE INVENTION

The field of the invention relates to processes for the preparation of polymorphic forms of salts of quetiapine. More particularly, it relates to the preparation of polymorphic forms of quetiapine hydrochloride designated as Forms A, B, and C. The invention also relates to pharmaceutical compositions that include the Form A, Form B, and Form C and use of the compositions for treating schizophrenia, acute manic episodes associated with bipolar I disorder. The invention also relates to the preparation of an oxalate salt of quetiapine and maleate salt of quetiapine.

### CROSS-REFERENCE TO RELATED APPLICATIONS

This application claims priority under 35 U.S.C. 119(a) from Indian Patent Application 1389/DEL/2005 filed on May 30, 2005, and incorporates the entirety herein by reference.

### BACKGROUND OF THE INVENTION

Quetiapine of Formula I is a psychotropic agent belonging to a chemical class of dibenzothiazepine derivatives. Chemically, quetiapine is 2-[2-(4-dibenzo[b,f][1,4]thiazepin-11-ylpiperazin-1-yl)ethoxy]ethanol. It is indicated for the treatment of schizophrenia, and acute manic episodes associated with bipolar I disorder.

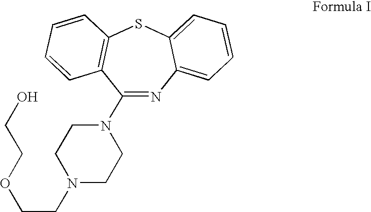

U.S. Pat. No. 4,879,288 discloses a process for the preparation of quetiapine or pharmaceutically acceptable salts thereof which includes hydrochloride, maleate, fumarate, citrate, phosphonate, methane sulphonate, and hemifumarate salt.

Several processes have been reported for the preparation of quetiapine or pharmaceutically acceptable salts thereof for example, in European patent EP 282,236; International (PCT) Publication Nos. WO 05/012274; 05/028457; 05/028458; and 05/028459.

International (PCT) Publication No. WO 01/55125 and 04/076431 discloses processes for the preparation of quetiapine hemifumarate.

International (PCT) Publication No. WO 05/014590 discloses processes for the preparation of Quetiapine freebase and quetiapine hemifumarate.

U.S. Pat. No. 6,372,734 provides a process for purification of crystalline quetiapine or pharmaceutically acceptable salts thereof which includes hydrochloride, maleate, fumarate, citrate, phosphonate, methane sulphonate, in particular the hemifumarate salt, which involves crystallising quetiapine freebase.

International (PCT) Publication No. WO 03/80065 discloses processes for the preparation of crystalline forms of quetiapine hemifumarate referred to as Form II and Form III, which are solvated polymorphic forms with dichloromethane and chloroform.

International (PCT) Publication No. WO 04/78735 discloses a process for the preparation of crystalline forms of quetiapine fumarate referred to as Form I and Form II. It also discloses processes for the preparation of amorphous quetiapine fumarate.

Salt formation of pharmaceutically active substance provides a means of altering the physicochemical and resultant biological characteristics of that substance without modifying its chemical structure. A salt form can have a dramatic influence on the properties of the drug, which includes yield, quantity of the crystalline structure, hygroscopicity, stability, solubility and the ease of formulation.

### SUMMARY OF THE INVENTION

In one general aspect there is provided an oxalate salt of queiapine.

The oxalate salt of quetiapine may have the X-ray diffraction pattern of FIG. 1 and infrared spectrum of FIG. 2.

In another general aspect there is provided a pharmaceutical composition that includes a therapeutically effective amount of the oxalate salt of quetiapine; and one or more pharmaceutically acceptable carriers, excipients or diluents.

In another general aspect there is provided a process for the preparation of an acid addition salt of quetiapine. The process includes contacting quetiapine fumarate in one or more solvents with a base to get quetiapine free base; treating the quetiapine free base with an acid; and isolating the acid addition salt of quetiapine by the removal of the solvents.

Removing the solvents may include, for example, one or more of filtration, filtration under vacuum, decantation and centrifugation. The process may include further forming of the product so obtained into a finished dosage form.

The process may include further drying of the product obtained.

In another general aspect there is provided a polymorphic Form A of quetiapine hydrochloride.

The Form A of quetiapine hydrochloride may have the X-ray diffraction pattern of FIG. 3 and infrared spectrum of FIG. 4.

In another general aspect there is provided a polymorphic Form B of quetiapine hydrochloride.

The Form B of quetiapine hydrochloride may have the X-ray diffraction pattern of FIG. 5 and infrared spectrum of FIG. 6.

In another general aspect there is provided a polymorphic Form C of quetiapine hydrochloride.

The Form C of quetiapine hydrochloride may have the X-ray diffraction pattern of FIG. 7 and infrared spectrum of FIG. 8.

In another general aspect there is provided amorphous form of quetiapine hydrochloride.

The amorphous form of quetiapine hydrochloride may have the X-ray diffraction pattern of FIG. 9.

In another general aspect there is provided a polymorphic Form I of quetiapine maleate.

The Form I of quetiapine maleate may have the X-ray diffraction pattern of FIG. 10 and infrared spectrum of FIG. 11.

In another general aspect there is provided a pharmaceutical composition that includes a therapeutically effective amount of the polymorphic Forms A, B, C, or amorphous form of quetiapine hydrochloride; and one or more pharmaceutically acceptable carriers, excipients or diluents.

In another general aspect there is provided a pharmaceutical composition that includes a therapeutically effective amount of the maleate salt of quetiapine; and one or more pharmaceutically acceptable carriers, excipients or diluents.

In another general aspect there is provided a method of treating schizophrenia and acute manic episodes associated with bipolar I disorder in a warm-blooded animal. The method includes providing a pharmaceutical composition to the warm-blooded animal, the pharmaceutical composition comprising the polymorphic Form A, Form B, Form C, or amorphous form of quetiapine hydrochloride, or the polymorphic Form I of quetiapine maleate or the oxalte salt of quetiapine.

The details of one or more embodiments of the invention are set forth in the description below. Other features, objects and advantages of the invention will be apparent from the description and claims.

### DESCRIPTION OF THE DRAWINGS

FIGS. 1a and 1b are an X-ray powder diffraction pattern of quetiapine oxalate and the associated values, respectively.

FIG. 2 is a Fourier Transform Infrared (FTIR) spectrum of quetiapine oxalate.

FIGS. 3a and 3B are an X-ray powder diffraction pattern of polymorphic Form A of quetiapine hydrochloride and the associated values, respectively.

FIG. 4 is a Fourier Transform Infrared (FTIR) spectrum of polymorphic Form A of quetiapine hydrochloride.

FIGS. 5a and 5b are an X-ray powder diffraction pattern of polymorphic Form B of quetiapine hydrochloride and the associated values, respectively.

FIG. 6 is a Fourier Transform Infrared (FTIR) spectrum of polymorphic Form B of quetiapine hydrochloride.

FIGS. 7a and 7b are an X-ray powder diffraction pattern of polymorphic Form C of quetiapine hydrochloride and the associated values, respectively.

FIG. 8 is a Fourier Transform Infrared (FTIR) spectrum of polymorphic Form C of quetiapine hydrochloride.

FIG. 9 is an X-ray powder diffraction pattern of amorphous form of quetiapine hydrochloride.

FIGS. 10a and 10b are an X-ray powder diffraction pattern of polymorphic Form I of quetiapine maleate and the associated values, respectively.

FIG. 11 is a Fourier Transform Infrared (FTIR) spectrum of polymorphic Form I of quetiapine maleate.

### DETAILED DESCRIPTION OF THE INVENTION

The present inventors have developed a process for the preparation of the oxalate salt of quetiapine. The oxalate salt of quetiapine is found to be stable and having improved solubility in water than the known hemifumarate salt.

The present inventors also have found new polymorphic forms of quetiapine hydrochloride. The new polymorphic forms are designated as Form A, Form B, Form C and an amorphous form. Further, the present inventors also have prepared a new crystalline polymorphic form of quetiapine maleate. These crystalline forms are more stable and have better solubility in water; thus offer distinct advantages over the known forms of hydrochloride or maleate.

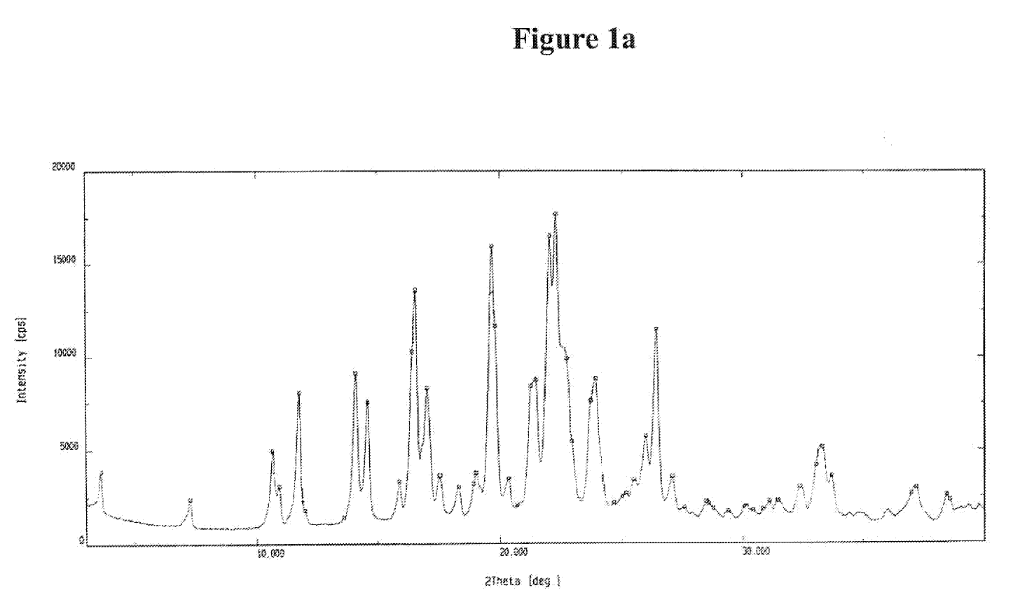

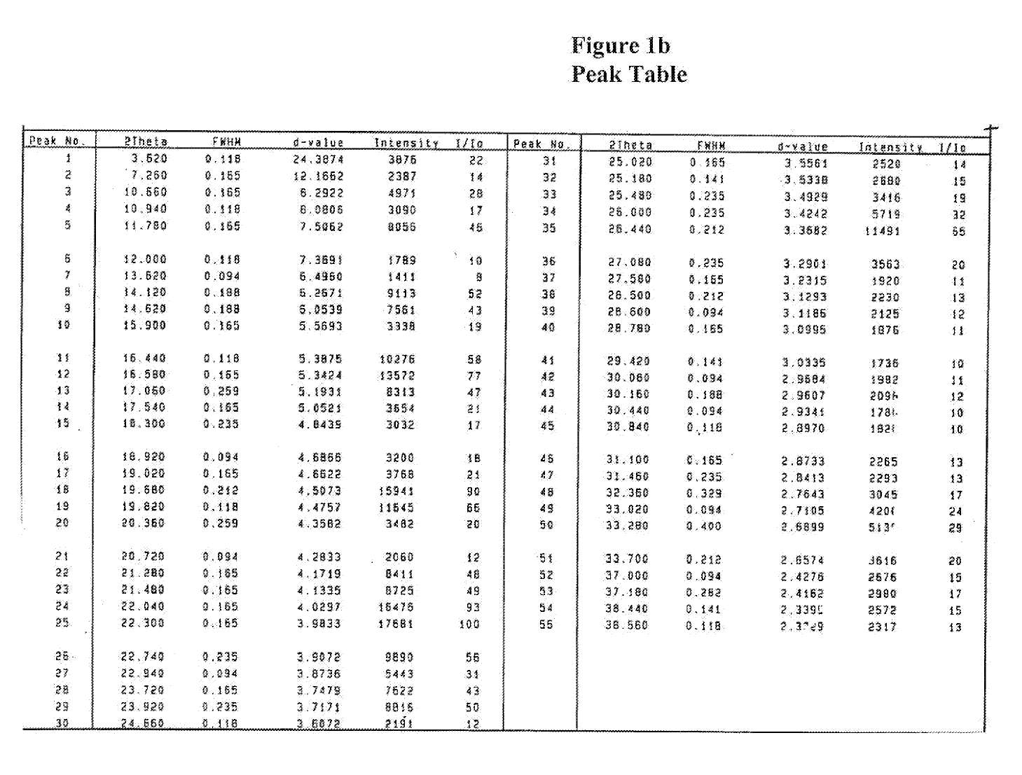

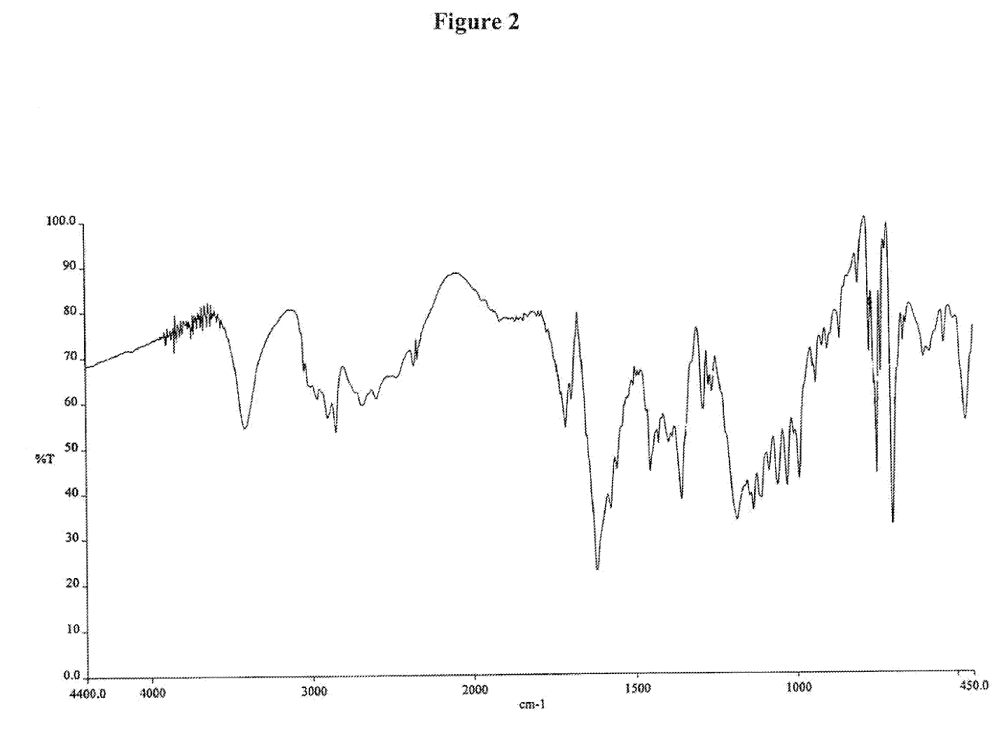

A first aspect of the present invention provides an oxalate salt of quetiapine. The oxalate salt exists in crystalline form having X-Ray Powder Diffraction (XRPD) pattern as depicted in FIG. 1 and Fourier Transform Infra-red (FTIR) spectrum as depicted in FIG. 2.

A second aspect of the invention provides a process for the preparation of acid addition salt of quetiapine, wherein the process includes the steps of:
a) contacting quetiapine fumarate in one or more solvents with a base to get quetiapine free base;
b) treating the quetiapine free base obtained in step a) with an acid; and
c) isolating the acid addition salt of quetiapine by the removal of the solvents.

The term “contacting” includes mixing, adding, slurrying, stirring or a combination thereof.

The quetiapine fumarate can be prepared by any of the methods known in the art including those described in U.S. Pat. Nos. 4,879,288; 6,372,734; and WO 05/012274; 05/028457; 05/028458 and 04/78735.

The quetiapine fumarate may be mixed in one or more solvents, including, for example, water, esters, lower alkanols, ethers, ketones, halogenated hydrocarbons or mixtures thereof.

Suitable alkanol solvents include methanol, ethanol, n-propanol, isopropanol and butanol. The ketone may include one or more of acetone, ethyl methyl ketone, methyl isobutyl ketone, and diisobutyl ketone. Examples of chlorinated hydrocarbons include dichloromethane, chloroform, and 1,2-dichloroethane. Examples of esters include ethyl acetate, n-propyl acetate, isopropyl acetate, and n-butyl acetate. Examples of ethers include diethyl ether, tetrahydrofuran, and the like. Mixtures of all of these solvents are also contemplated.

The base can be selected from inorganic or organic bases such as alkali and alkaline earth metal hydroxide or alkoxide, ammonia, triethylamine, dicyclohexylamine amine or diisopropylamine, and the like.

The free base may be obtained by concentrating the solvent to get an oily mass. The oily mass may be dissolved in an organic solvent selected from esters, lower alkanols, ethers, ketones, halogenated hydrocarbons, or mixtures thereof. The resultant mixture may be acidified with a suitable acid. The precipitated solids are filtered, washed and dried to yield Quetipine acid addition salt.

The acid used is inorganic or organic acid. Suitable acids include hydrochloric acid, hydrobromic acid, sulphuric acid, phosphoric acid, oxalic acid, maleic acid, fumaric acid, succinic acid, citric acid, methane sulphonic acid, acetic acid, and the like.

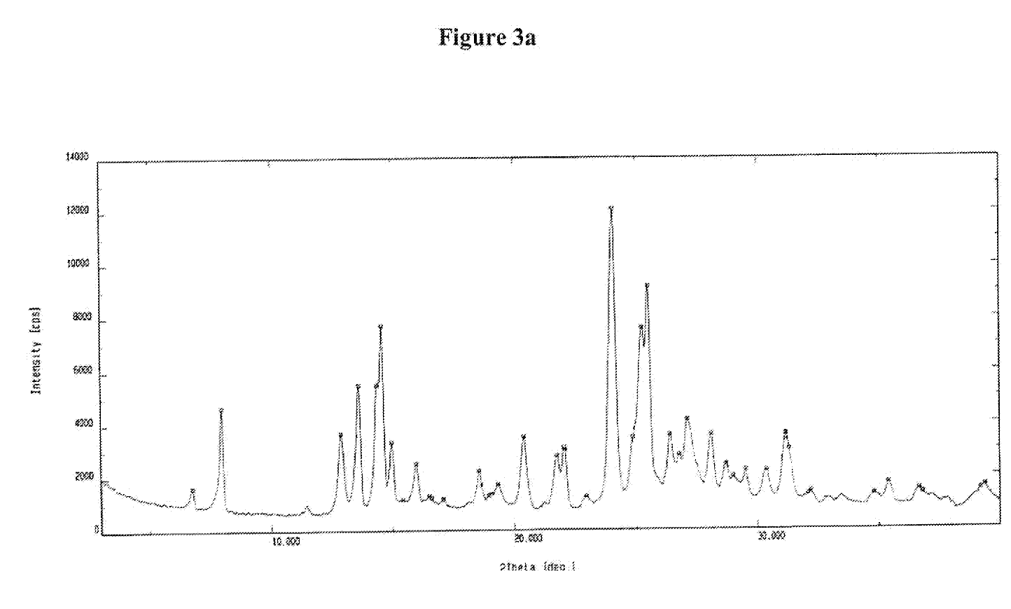

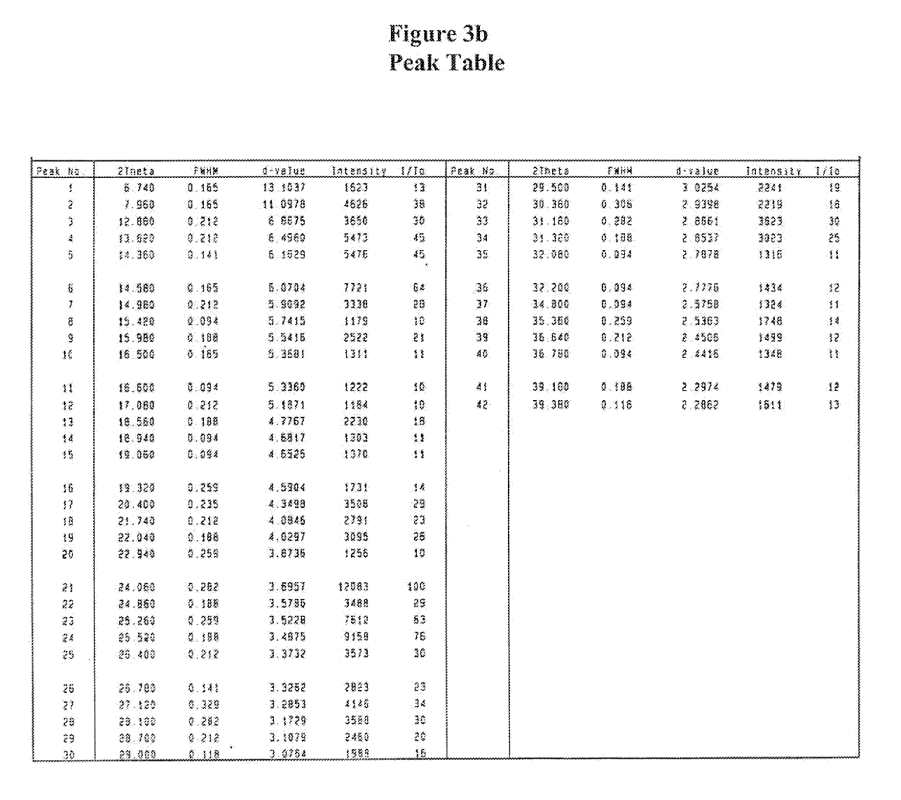

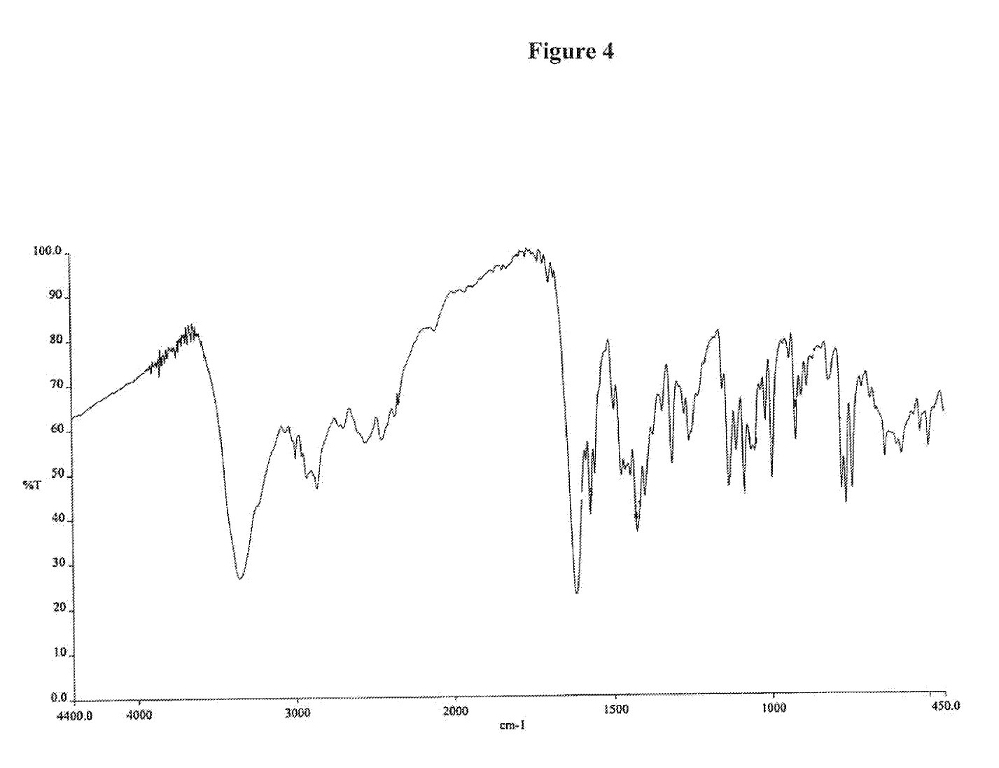

A third aspect of the present invention provides polymorphic Form A of quetiapine hydrochloride having a characteristic XRD pattern as depicted in FIG. 3 and the FTIR spectrum in potassium bromide as depicted in FIG. 4. The polymorphic Form A may have characteristic absorption bands at 2-theta values of about 6.74, 7.96, 12.88, 13.62, 14.36, 14.58, 14.98, 15.98, 18.56, 20.40, 21.74, 22.04, 24.06, 25.26, 25.52, 26.40, 27.12, 28.10, 30.38, and 31.18.

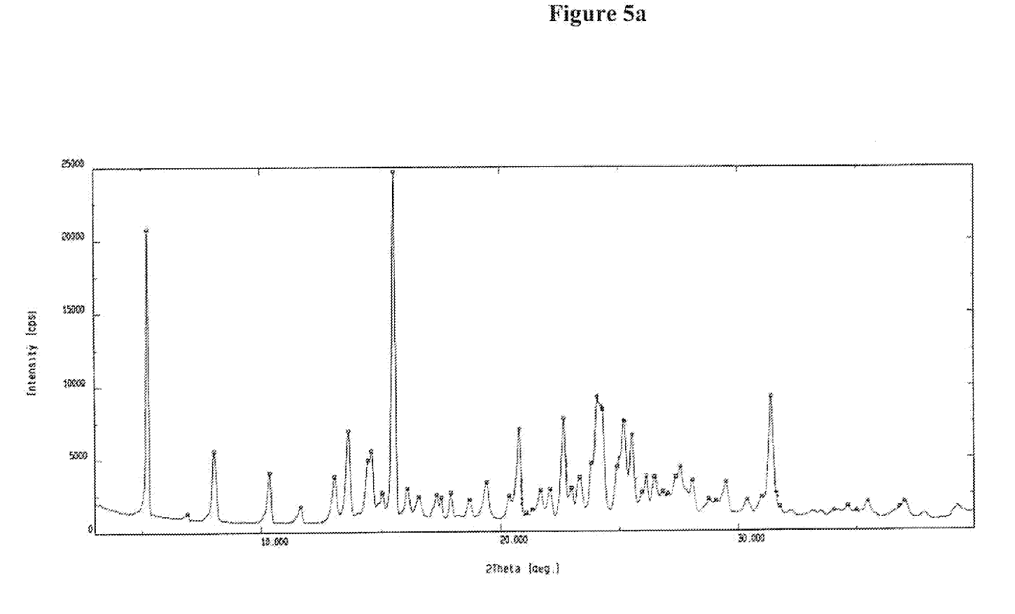

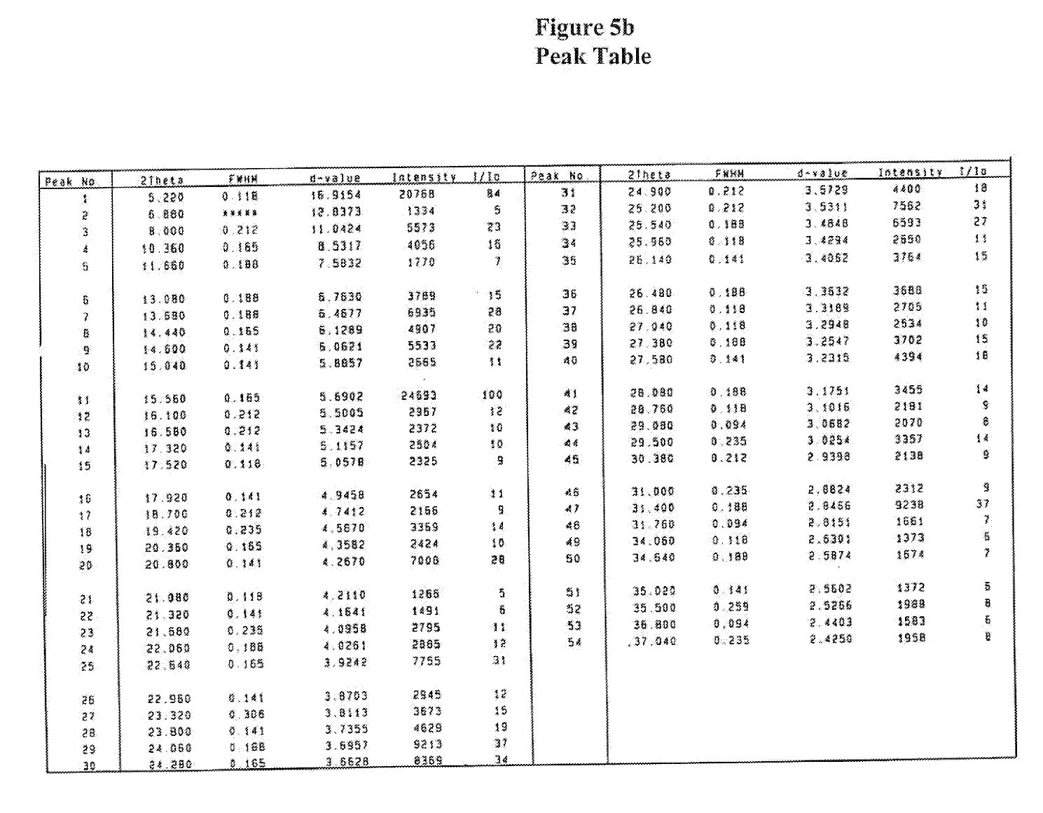

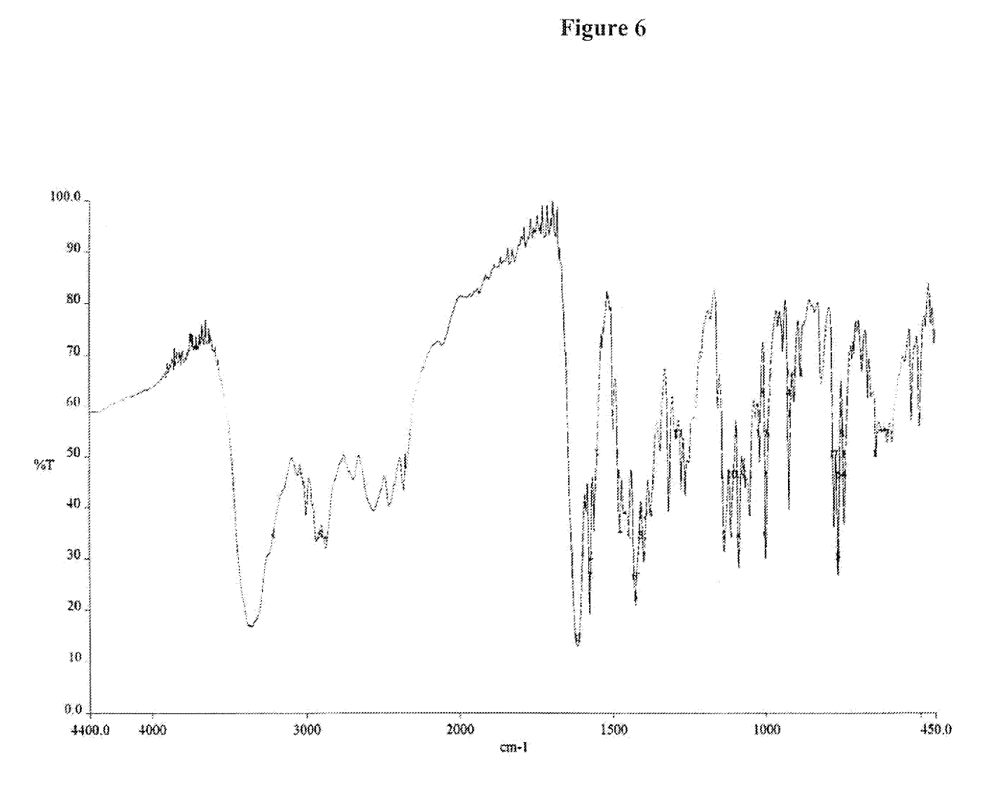

A fourth aspect of the present invention provides polymorphic Form B of quetiapine hydrochloride having a characteristic XRD pattern as depicted in FIG. 5 and absorption bands at 2-theta values of about 5.22, 6.88, 8.00, 10.36, 11.66, 13.08, 13.68, 14.80, 15.56, 16.10, 16.58, 17.32, 17.98, 18.70, 19.42, 20.80, 22.64, 24.06, 25.20, 25.54, and 31.40. The Form B of quetiapine hydrochloride may have the FTIR spectrum in potassium bromide as depicted in FIG. 6.

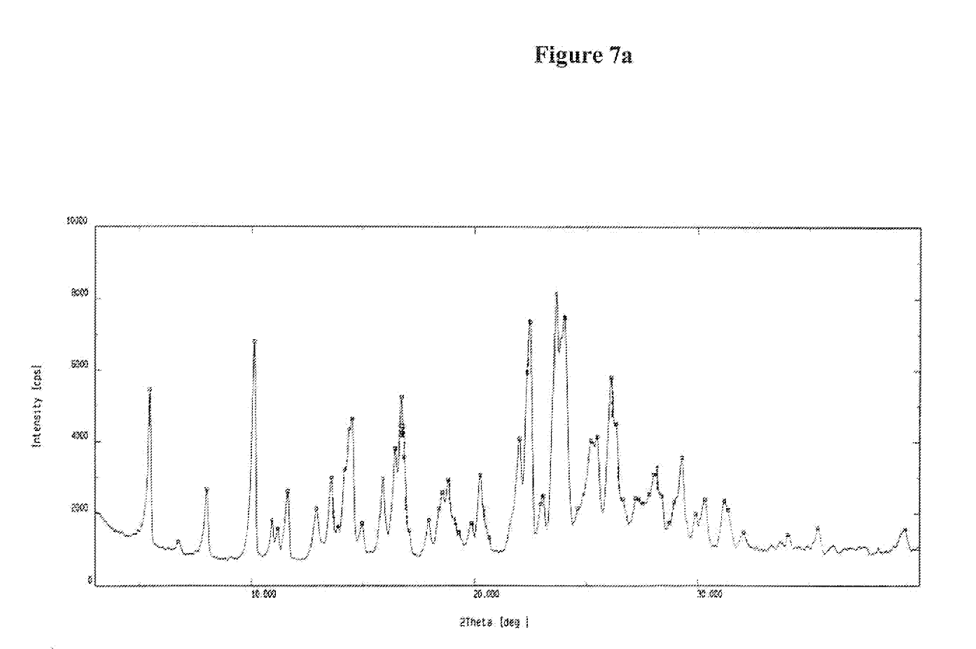

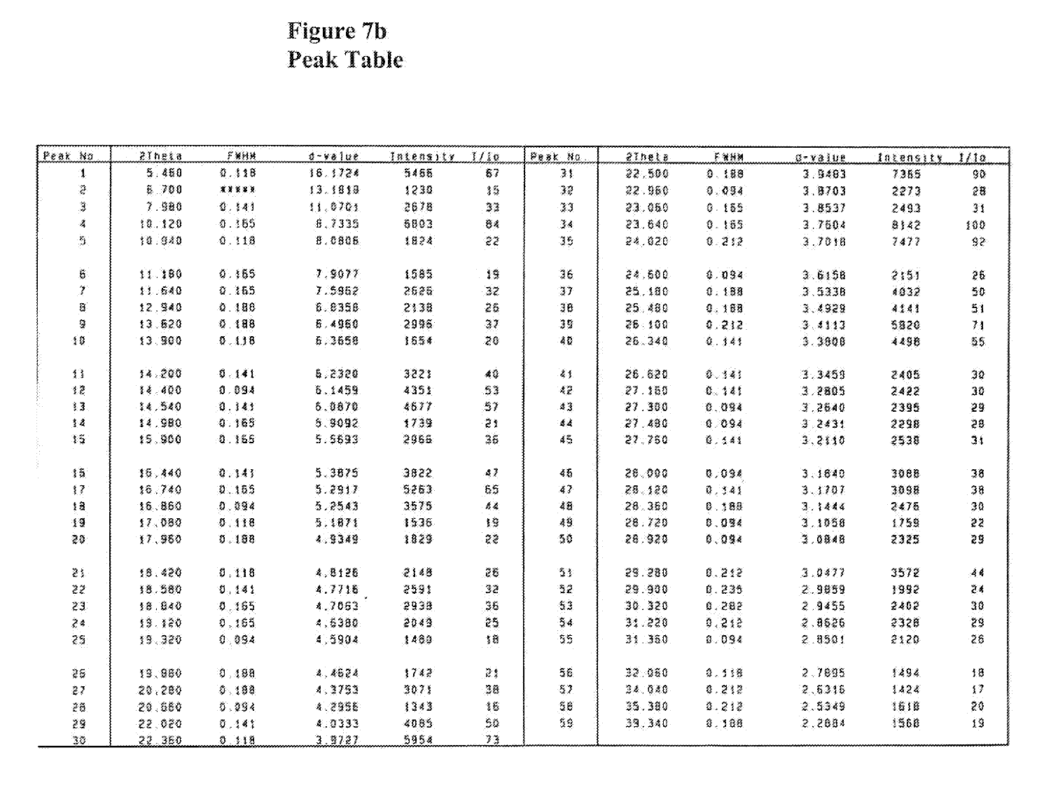

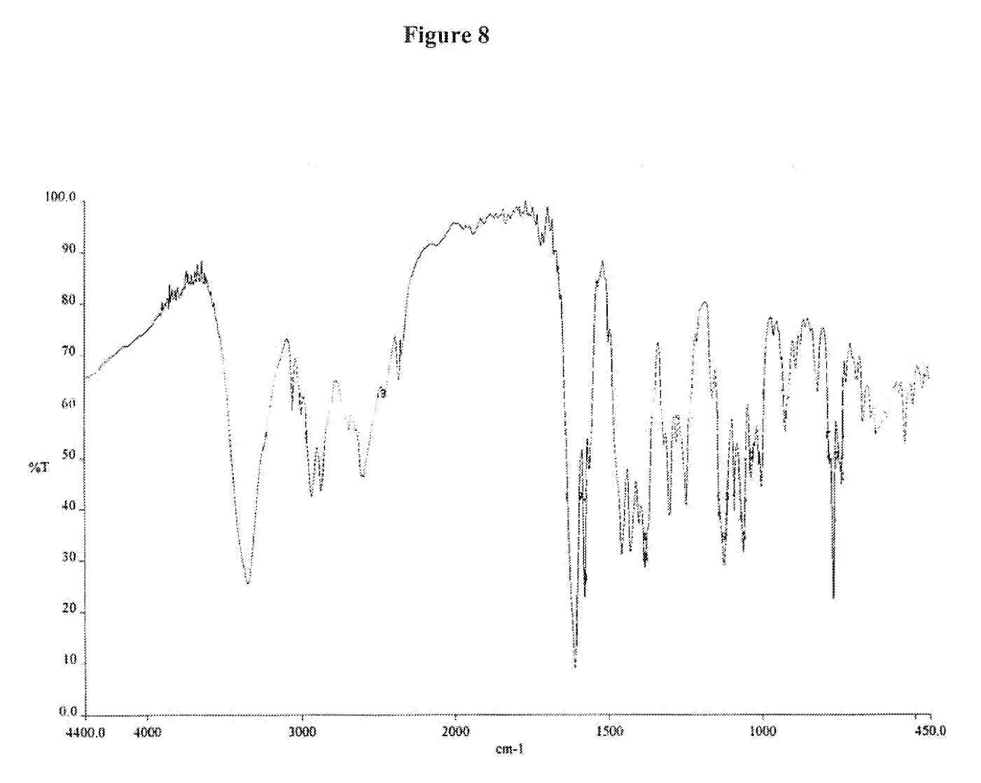

A fifth aspect of the present invention provides polymorphic Form C of quetiapine hydrochloride having XRD pattern as depicted in FIG. 7 and FTIR spectrum as depicted in FIG. 8. The Form C may have characteristic absorption bands at 2-theta values of about 5.46, 7.98, 10.12, 10.94, 11.64, 12.94, 13.62, 14.20, 14.54, 15.90, 16.44, 16.74, 17.96, 18.84, 20.28, 22.02, 22.50, 23.64, 24.02, 25.18, 25.48, 26.10, 29.28, and 30.32.

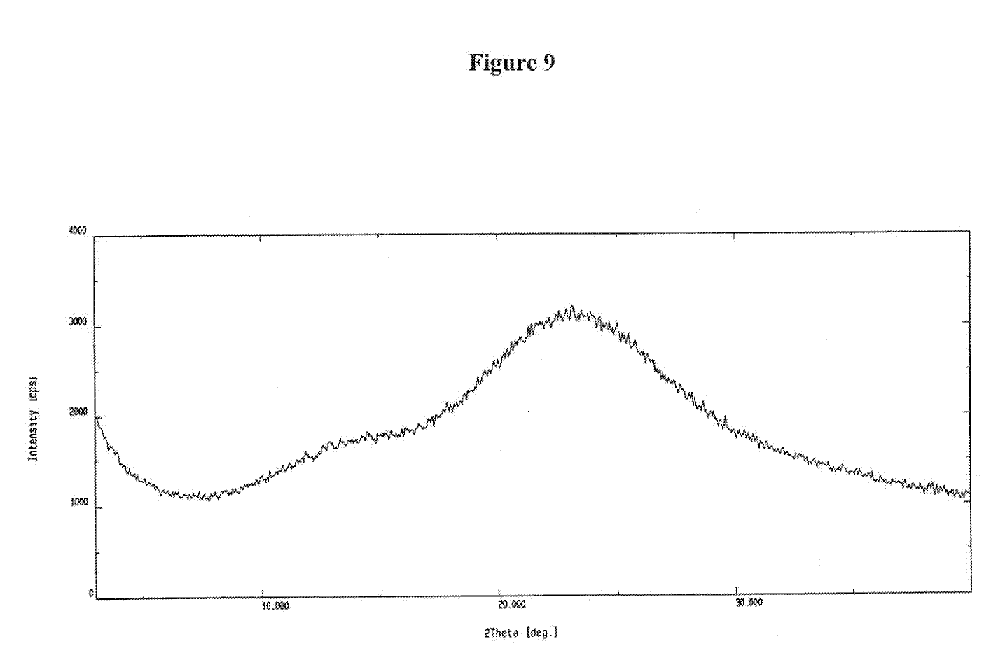

A sixth aspect of the present invention provides an amorphous form of quetiapine hydrochloride having XRD pattern as depicted in FIG. 9. The amorphous quetiapine hydrochloride is found to be stable, non-hygroscopic and more soluble in water as compared to any other crystalline form.

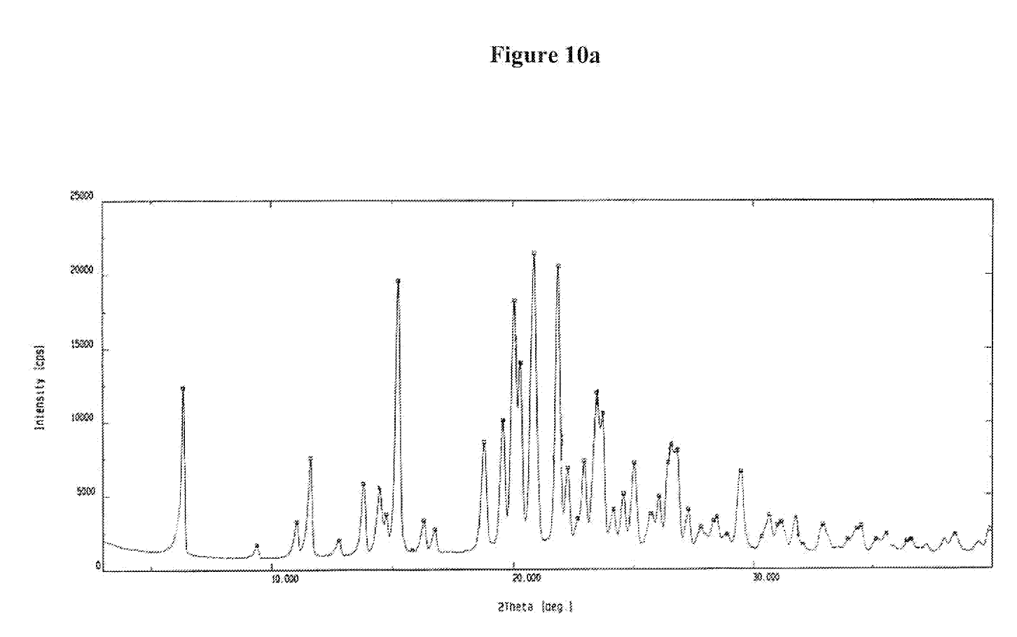

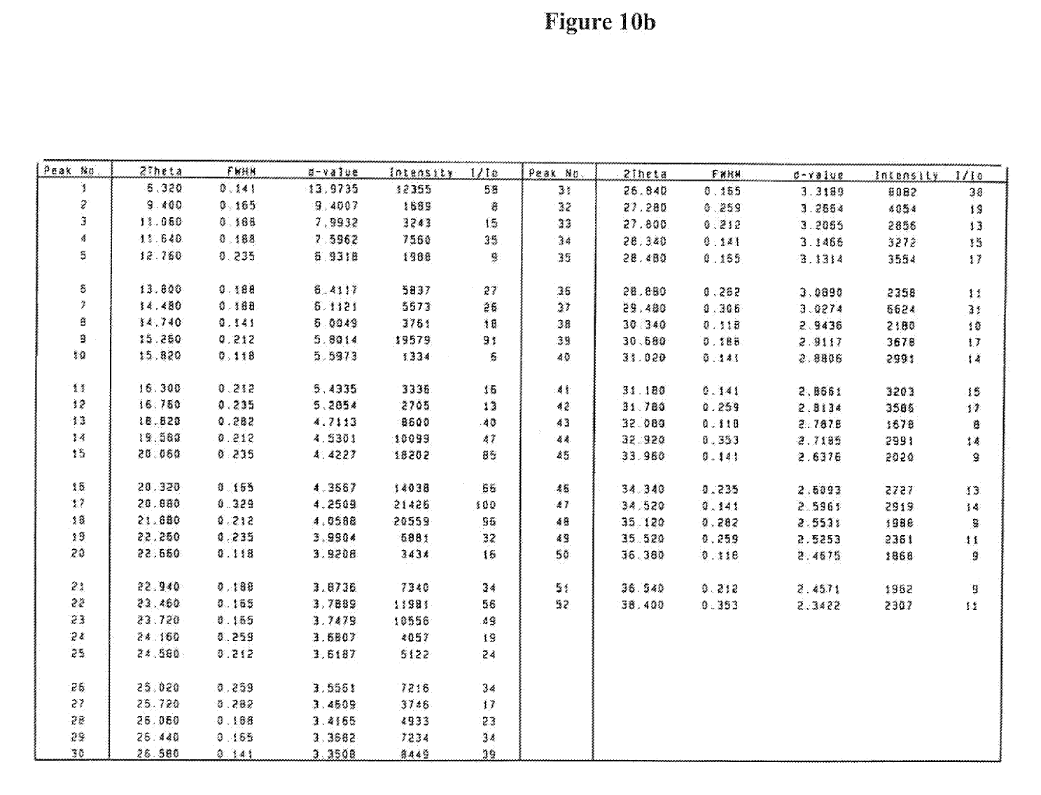

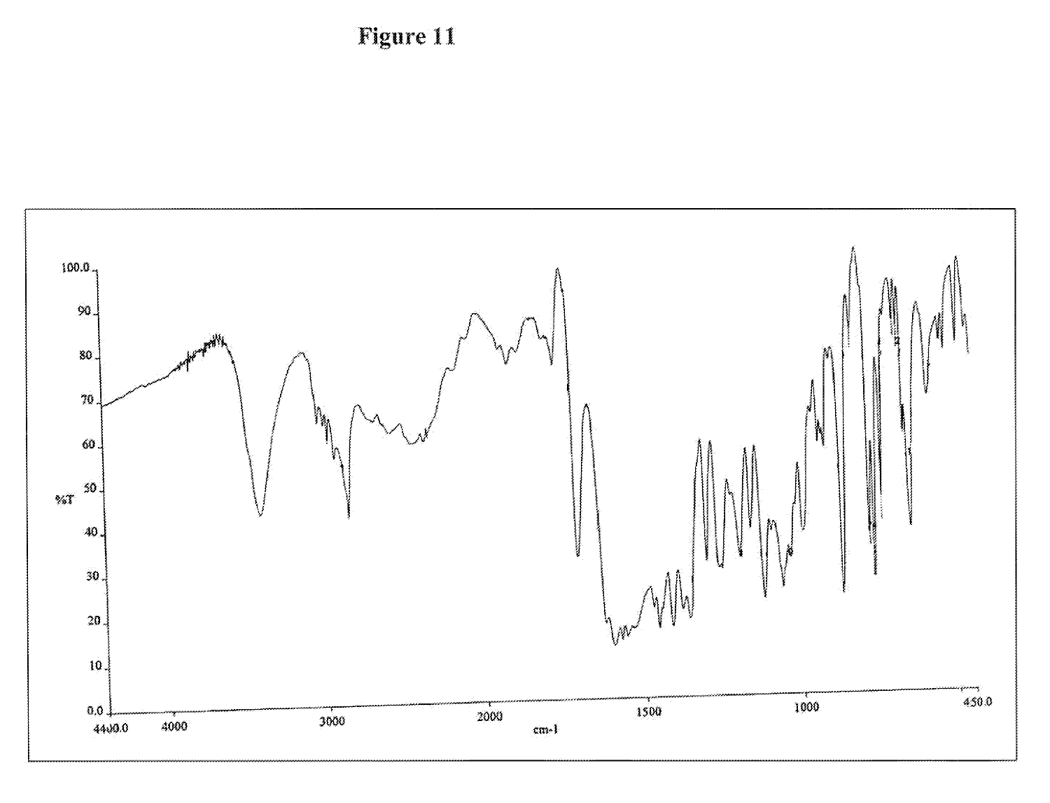

A seventh aspect of the present invention provides polymorphic Form I of quetiapine maleate having XRD pattern as depicted in FIG. 10 with characteristic absorption bands at 2-theta values of about 6.32, 11.06, 11.64, 12.76, 13.80, 14.48, 15.26, 16.30, 16.76, 18.82, 19.56, 20.06, 20.32, 20.88, 21.88, 22.26, 22.94, 23.46, 23.72, 24.58, 25.02, 26.06, 26.58, and 29.48. The polymorphic Form I of quetiapine maleate may have the FTIR spectrum in potassium bromide as depicted in FIG. 11.

An eighth aspect of the present invention provides a pharmaceutical composition comprising Form A, Form B, Form C or the amorphous form of quetiapine hydrochloride along with pharmaceutically acceptable carrier(s) and/or excipient(s). The pharmaceutical compositions include one or more oral dosage forms such as tablets, capsules, liquid orals, suspensions and the like, as well as topical dosage forms such as creams, lotions, ointments and the like.

A ninth aspect of the present invention provides a pharmaceutical composition comprising quetiapine oxalate along with pharmaceutically acceptable carrier(s) and/or excipient(s).

A tenth aspect of the present invention provides a pharmaceutical composition comprising or polymorphic Form I of quetiapine maleate along with pharmaceutically acceptable carrier(s) and/or excipient(s).

An eleventh aspect of the present invention provides a method of treating schizophrenia, acute manic episodes associated with bipolar I disorder comprising administering to a mammal in need thereof a therapeutically effective amount of Form A, Form B, Form C or amorphous form of quetiapine hydrochloride or quetiapine oxalate or polymorphic Form I of quetiapine maleate.

For the purpose of this disclosure, a warm-blooded animal is a member of the animal kingdom possessed of a hemostatic mechanism and includes mammals and birds.

Powder XRD of the samples were determined by using X-Ray Difractometer, Rigaku Corporation, RU-H3R, Goniometer CN2155A3, X-Ray tube with Cu target anode, Divergence slits 1 0, Receiving slit 0.15 mm, Scatter slit 1°, Power: 40 KV, 100 mA, Scanning speed: 2 deg/min step: 0.02 deg, Wave length: 1.5406 A

FTIR of the samples were determined by using Instrument: Perkin Elmer, 16 PC, SCAN: 16 scans, 4.0 cm⁻¹, according to the USP 25, general test methods page 1920, infrared absorption spectrum by potassium bromide pellet method.

The following examples are provided merely to show various implementations of the inventions and should not be construed as limiting the scope of the claims.

### EXAMPLE 1

### Preparation of Quetiapine Oxalate

Quetiapine fumarate (50 g) was charged to water (500 ml) and ethyl acetate (500 ml) at room temperature. Ammonia solution was added with stirring till pH 10. The organic phase was separated and washed with water (500 ml×2). The organic layer was concentrated to oily mass under reduced pressure at 45-50° C. The oil so obtained was dissolved in acetone (750 ml) and heated to 45-48° C. Oxalic acid (14.9 g in acetone (375 ml) at 45° C.) was added in one lot and stirred for 30 minutes at 45-48° C. The mixture was further stirred at room temperature for one hour, filtered, washed with acetone (250 ml) and dried at 45° C. under reduced pressure to obtain the title compound.

Yield: 46.0 g

### EXAMPLE 2

### Preparation of Form A of Quetiapine Hydrochloride

Quetiapine fumarate (50 g) was charged to water (500 ml) and ethyl acetate (500 ml) at room temperature. Ammonia solution was added with stirring till pH 10. The organic phase was separated and washed with water (500 ml×2). The organic layer was concentrated to oily mass under reduced pressure at 45-50° C. The oil so obtained was dissolved in isopropanol (200 ml) and water (12.5 ml). Concentrated hydrochloric acid (23.5 ml) was added at room temperature. Ethyl acetate (500 ml) was added in 15 minutes to the solid so obtained, and the mixture was stirred and filtered. The solid was dried at 45-50° C. under vacuum to obtain the title compound.

### EXAMPLE 3

### Preparation of Form B of Quetiapine Hydrochloride

Quetiapine fumarate (1 g) was charged to water (10 ml) and ethyl acetate (10 ml) at room temperature. Ammonia solution was added with stirring till pH 10. The organic phase was separated and washed with water (10 ml×2). The organic layer was concentrated to oily mass under reduced pressure at 45-50° C. The oil so obtained was dissolved in isopropanol (4 ml). It was acidified with isopropanol-hydrochloride. To this mixture, ethyl acetate (20 ml) was added and stirred. Again, ethyl acetate (10 ml) was added and stirred for one hour. The solid so obtained was filtered, washed with ethyl acetate and dried at 45-50° C. under vacuum to obtain the title compound.

Yield: 0.8 g

### EXAMPLE 4

### Preparation of Form C of Quetiapine Hydrochloride

Quetiapine fumarate (1 g) was charged to water (10 ml) and ethyl acetate (10 ml) at room temperature. Ammonia solution was added with stirring till pH 10. The organic phase was separated and washed with water (10 ml×2). The organic layer was concentrated to oily mass under reduced pressure at 45-50° C. The oily mass so obtained was dissolved in isopropanol (4 ml) and acidified with concentrated hydrochloride. To this mixture, ethyl acetate (20 ml) was added drop wise. The mixture was further stirred for one hour and left over night. The solid obtained was filtered, dried at 45-50° C. under vacuum to obtain the title compound.

Yield: 0.66 g

### EXAMPLE 5

### Preparation of Amorphous Form of Quetiapine Hydrochloride

Quetiapine fumarate (20 g) was charged to water (200 ml) and ethyl acetate (200 ml) at room temperature. Ammonia solution was added with stirring till pH 10. The organic phase was separated and washed with water (200 ml×2). The organic layer was concentrated to oily mass under reduced pressure at 45-50° C. The obtained oily mass was dissolved in ethyl acetate (200 ml) and acidified with dry hydrochloride gas till pH 1.0 at 10° C. The sticky mass was stirred for one hour, filtered and dried at 45-50° C. under vacuum to obtain the title compound.

Yield: 17.5 g

### EXAMPLE 6

### Preparation of Form I of Quetiapine Maleate

Quetiapine fumarate (50 g) was charged to water (500 ml) and ethyl acetate (500 ml) at room temperature. Ammonia solution was added with stirring till pH 10. The organic phase was separated and washed with water (500 ml×2). The organic layer was concentrated to oily mass under reduced pressure at 45-50° C. The oil so obtained was dissolved in ethyl acetate (500 ml) and heated to 60-65° C. Maleic acid solution (13.05 g in 420 ml ethyl acetate at 50-60° C.) was added in one lot and stirred for 10 minutes at 65° C. and cooled to room temperature. The solid obtained was filtered, washed with ethyl acetate (100 ml×2) and dried at 40° C. under reduced pressure to obtain the title compound.

While the present invention has been described in terms of its specific embodiments, certain modifications and equivalents will be apparent to those skilled in the art and are intended to be included within the scope of the present invention. For example, the compounds described herein can be formulated into dosage forms that are suitable for administering to patients in need of the compound for treating a medical condition for which the compound is indicated, approved, or otherwise beneficial. Specifically, the Forms A, B, C, and amorphous form of quetiapine hydrochloride, oxalate salt of quetiapine and Form I of quetiapine maleate can be formulated with one or more pharmaceutically acceptable excipients and/or with one or more active ingredients into a dosage form and administered to treat schizophrenia and acute manic episodes associate with bipolar I disorder.

We claim:

1. An oxalate salt of quetiapine.

2. A process for the preparation of an acid addition salt of quetiapine, the process comprising:
a) contacting quetiapine fumarate in one or more solvents with a base to get quetiapine free base;
b) treating the quetiapine free base with an acid; and
c) isolating the acid addition salt of quetiapine by the removal of the solvents.

3. The process according to claim 2, wherein the acid comprises one or more of hydrochloric acid, hydrobromic acid, sulphuric acid, phosphoric acid, oxalic acid, maleic acid, fumaric acid, succinic acid, citric acid, methane sulphonic acid, and acetic acid.

4. A polymorphic Form A of quetiapine hydrochloride having characteristic X-ray diffraction peaks at 2-theta values of about 6.74, 7.96, 12.88, 13.62, 14.58, 14.98, 15.98, 18.56, 20.40, 22.04, 24.06, 25.52, 26.40, 27.12, 28.10, and 30.38.
